(19) United States
(12) Patent Application Publication
(10) Pub. No.: US 2011/0224205 A1
(43) Pub. Date: Sep. 15, 2011

(54) USE OF CURCUMIN OR ITS ANALOGUES IN CANCER THERAPY UTILIZING EPIDERMAL GROWTH FACTOR RECEPTOR TYROSINE KINASE INHIBITOR

(75) Inventors: Huei-Wen Chen, Taipei City (TW); Jen-Yi Lee, Taichung City (TW); Pan-Chyr Yang, Taipei City (TW); Sung-Liang Yu, Taipei City (TW); Jian-Wei Chen, Taichung City (TW); Chih-Hsin Yang, Taipei City (TW); Chao-Chi Ho, Taipei City (TW); Kuo-Hsiung Lee, Chapel Hill, NC (US); Yufeng Jane Tseng, Taipei City (TW); Gee-Chen Chang, Taichung City (TW)

(73) Assignee: NATIONAL TAIWAN UNIVERSITY, TAIPEI CITY (TW)

(21) Appl. No.: 13/025,798

(22) Filed: Feb. 11, 2011

Related U.S. Application Data
(60) Provisional application No. 61/303,593, filed on Feb. 11, 2010.

(51) Int. Cl. A61K 31/5377; A61K 31/12; A61P 35/00

(52) U.S. Cl. 514/234.5; 514/679

(57) ABSTRACT

Provided is combined use of an epidermal growth factor receptor tyrosine kinase inhibitor (EGFR-TKI) and curcumin or its analogue in cancer therapy, which reduces side effects resulting from the EGFR-TKI and reduces doses of the EGFR-TKI needed for the therapy, particular in a patient resistant to the treatment with the EGFR-TKI alone.

### RELATED APPLICATION

This application claims priority to U.S. Provisional Application No. 61/303,593, filed on Feb. 11, 2010, the content of which is hereby incorporated by reference in its entirety.

### FIELD OF THE INVENTION

The present invention relates to use of curcumin or its analogues in cancer therapy utilizing an epidermal growth factor receptor tyrosine kinase inhibitor (EGFR-TKI), which reduces side effects resulting from the therapy and reduces doses of the EGFR-TKI needed for the therapy, particular in a patient resistant to treatment with the EGFR-TKI alone.

### BACKGROUND OF THE INVENTION

Cancer is the leading cause of death in the world. Recently, so called “target therapy” has been developed and agents that selectively target epidermal growth factor receptor (EGFR) have been shown to be of benefit clinically (Cancer Research 2004; 64(15):5355-62.). The EGFR pathway is a key driver in the regulation of cell growth and differentiation and acts via regulating the phosphorylation of intrinsic tyrosine kinases (Cancer Research 2003; 63(1):1-5). Over-expression of EGFR has been reported to occur in various malignant cells and is correlated with a poor prognosis (Oncologist 2004; 9(1):58-67). However, some critical issues still remain to limit the use of these agents.

Gefitinib (Iressa®), an orally active EGFR-TKI, is the first selective small molecular agent approved for non-small cell lung cancer (NSCLC) treatment (Lung Cancer 2003; 41 Suppl 1:S9-14; and Expert Review of Anticancer Therapy 2004; 4(1):5-17). Previous studies involving a multi-institutional clinical trial have been showed that the response to gefitinib is better in Asian patients compared to Caucasian patients and that women who are non-smokers and have adenocarcinoma are the most likely to benefit the most (Proceedings of the National Academy of Sciences of the United States of America 2004; 101(36):13306-11; and Lancet 2005; 366(9496):1527-37). Recent studies have indicated that in-frame deletions (ΔE746-A750) of exon 19 and L858R substitution in exon 21 of EGFR in NSCLC are highly correlated with gefitinib sensitivity (New England Journal of Medicine 2004; 350(21):2129-39; Science 2004; 304(5676):1497-500; and Oncologist 2008; 13(12):1276-84). However, the EGFR gene mutation rate of NSCLC patients has been found to range from 10% to 15% in Caucasians and from 30% to 40% in Asians (Clinical Cancer Research 2008; 14(10):2895-9; and Journal of the National Cancer Institute 2005; 97(5):339-46). Patients with a wild-type EGFR are still prominent in all NSCLC cases worldwide and this population shows a relatively poor response to gefitinib treatment. In addition, acquired resistance caused by a second site substitution, T790M in EGFR within exon 20, results in poor gefitinib activity (New England Journal of Medicine 2005; 352(8):786-92; and PLoS Medicine/Public Library of Science 2005; 2(3):e73).

Side effects are another limiting factor for the use of EGFR-TKIs. Gefitinib is known to cause side effects such as diarrhea (Journal of Clinical Oncology 2003; 21(12):2237-46; and Clinical Cancer Research 2004; 10(4):1212-8) and skin rash. The frequency of diarrhea caused by gefitinib was 67% in the 500 mg/day dose group and 48% in the 250 mg/day dose group during the clinical trials. A recent case report has indicated the concomitant use of EGFR-TKI and radiotherapy can cause unexpected toxicity and fatal diarrhea in a metastatic NSCLC patient (Lung Cancer 2008; 61(2):270-3). These adverse effects may lead to physical and psychosocial discomfort that can result in dose reduction or treatment interruption.

### BRIEF SUMMARY OF THE INVENTION

The present invention is based on the finding that curcumin is a potential agent to reduce side effects resulting from EGFR-TKI treatment and reduce doses of an EGFR-TKI needed for cancer therapy with the EGFR-TKI, particular in a patient resistant to treatment with the EGFR-TKI alone.

Accordingly, in one aspect, the present invention provides a method for reducing side effects resulting from treatment using an epidermal growth factor receptor tyrosine kinase inhibitor (EGFR-TKI), comprising administering curcumin or its analogue to a patient undergoing such treatment in an amount effective to reduce the side effects. In one embodiment, the side effects are EGFR-TKI induced adverse gastrointestinal effects, such as intestinal cell damage or growth inhibition.

In another aspect, the present invention provides a method for administering an epidermal growth factor receptor tyrosine kinase inhibitor (EGFR-TKI) to a patient in need of a cancer therapy using the EGFR-TKI, comprising administering to the patient a reduced dose of the EGFR-TKI in combination with curcumin or its analogue while efficacy of the EGFR-TKI with respect to the cancer therapy is substantially maintained as compared to that achieved with a standard dose of the EGFR-TKI without administration of the curcumin or its analogue. In one embodiment, the patient is diagnosed as EGFR-TKI resistant. In one embodiment, the reduced dose is about 50% or less of the standard dose of the EGFR-TKI.

In one embodiment, the patient to be treated is afflicted with non-small-cell lung cancer (NSCLC). In another embodiment, the patient to be treated is resistant to EGFR-TKI.

In one embodiment, the curcumin analogues are selected from the group consisting of:

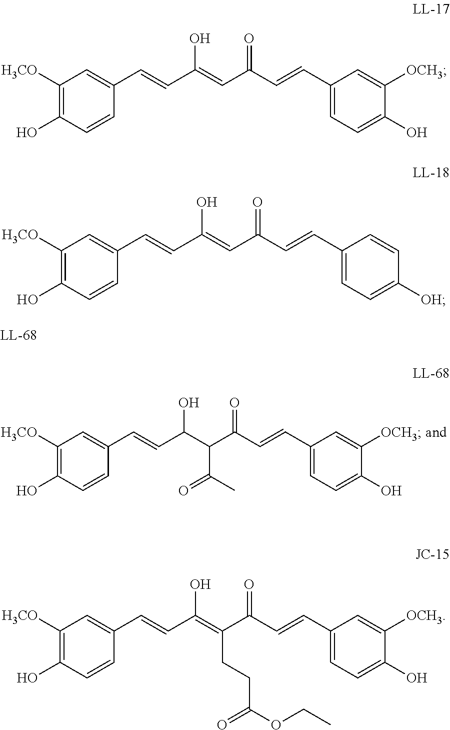

In one embodiment, the EGFR-TKI is gefitinib (N-(3-Chloro-4-fluoro-phenyl)-7-methoxy-6-(3-morpholin-4-ylpropoxy)quinazolin-4-amine).

In one embodiment, the curcumin or its analogue is administered concurrently with the EGFR-TKI.

Also provided is a method for treating a cancer patient with resistance to an EGFR-TKI, which comprises jointly administering to said patient an effective amount of the EGFR-TKI with curcumin or its analogue.

The various embodiments of the present invention are described in details below. Other characteristics of the present invention will be clearly presented by the following detailed descriptions and drawings about the various embodiments and claims.

It is believed that a person of ordinary knowledge in the art where the present invention belongs can utilize the present invention to its broadest scope based on the descriptions herein with no need of further illustration. Therefore, the following descriptions should be understood as of demonstrative purpose instead of limitative in any way to the scope of the present invention.

### BRIEF DESCRIPTION OF THE SEVERAL VIEWS OF THE DRAWINGS

For the purpose of illustrating the invention, there are shown in the drawings embodiments which are presently preferred. It should be understood, however, that the invention is not limited to the preferred embodiments shown.

In the drawings:

FIG. 1 shows that curcumin potentially inhibits cell proliferation and decreases ligand-induced EGFR signaling activation in gefitinib-resistant NSCLC cells. A, left, MTT assay results shows susceptibility to gefitinib in all six lung adenocarcinoma cell lines; right, MTT assay shows that curcumin causes the dose-dependent suppression of cell proliferation in the five gefitinib-resistant NSCLC cell lines. Columns, mean (n=6); bars, SD. Data are representative of two independent experiments. B, Western blotting shows that curcumin decreases EGF (20 ng/ml)-induced EGFR expression, pEGFR levels, AKT expression, pAKT levels and cycline D1 expression in CL1-5, A549 and H1975 cell lines in a dose-dependent manner (1-20 μM). Data are representative of two independent experiments with β-actin used as the internal control. C, Real-time quantitative RT-PCR reveals that EGFR mRNA expression is reduced by curcumin in CL1-5 cells in a concentration-dependent manner (1-20 μM) after 24 h incubation. Columns, mean (n=3); bars, SD. *, p<0.05, statistically significantly compared with the vehicle-treated control. Data are representative of three independent experiments.

FIG. 2 shows the binding of curcumin on the tyrosin phosphorylation site of EGFR with T790M resistant mutation.

FIG. 3 shows that curcumin accelerates EGFR degradation and downregulates EGFR protein level. A, Western blotting shows that curcumin was able to inhibit the EGFR protein expression in a dose-dependent manner while in the cells pre-treated with MG132, the EGFR protein level was recovered. B, Immunoprecipitation assay shows that gefitinib (1 μM) and curcumin (10 μM) was able to increase the EGFR protein ubiquitination compare to the other single treatment group.

FIG. 4 shows the enhancement of anti-cell proliferation and blockage of the EGFR signaling activation caused by gefitinib using curcumin in vitro. A, MTT assays show that curcumin enhances the anti-proliferative effect of gefitinib in five gefitinib-resistant lung adenocarcinoma cell lines. Columns, mean (n=6); bars, SD. **, p<0.01. Data are representative of two independent experiments. B, Western blotting shows that curcumin increases blockage of EGF (20 ng/ml)-induced EGFR and further reduces the pEGFR, AKT and pAKT protein expression levels after gefitinib treatment of CL1-5, A549 and H1299 NSCLC cell lines. Data are representative of two independent experiments with β-actin used as the internal control.

FIG. 5 shows that curcumin enhances the apoptotic effects and colony formation inhibition of gefitinib in NSCLC cells in vitro. A, The Annexin V-FITC apoptosis assay shows that curcumin enhances the apoptotic effect of gefitinib; upper, CL1-5 cells were treated with the agents indicated. The x axis is Annexin-V-FITC, and they axis is PI (propidium iodide) for all graphs presented; lower, CL1-5, A549 and H1975 cells undergoing apoptosis were counted; the Annexin-V+ and PI− (early apoptosis) and Annexin-V+ and PI+ (late apoptosis) levels of total cells are found in the lower right and upper right quadrants. Columns, mean (n=3); bars, SD. *, p<0.05, **, p<0.01 and p=0.331 (15 μM curcumin versus 1 μM gefitinib plus 15 μM curcumin). Data are representative of triplicate independent experiments. B, The colony formation assay shows that curcumin enhances the colony inhibitory ability of gefitinib in CL1-5, A549 and H1975 lung adenocarcinoma cells. Cell treatment with agents is indicated; V: vehicle control, G1, G5, G10, G15, C1, C5, C10, C15, G1+C1, G1+C5, G1+C10 and G1+C15: G and C indicated gefitinib and curcumin, and number showed the concentration (μM) of the agents. Colonies were counted after 2 wk. Columns, mean (n=3); bars, SD. Data are representative of two independent experiments.

FIG. 6 shows that curcumin enhances the antitumor activity of gefitinib in vivo. A, in vivo experimental protocol presented as a scheme. B, 10⁶ CL1-5 lung adenocarcinoma cells were implanted s.c. in SCID mice and the tumor volumes of the five groups were monitored over time. Points, mean with at least six mice per group; bars, SD. C, upper, photographs of the s.c. tumor excised from mice indicated the curcumin enhanced activity of gefitinib against tumor growth compare to the alone treatment groups; lower, tumor volume on the last day of the experiment. Columns, mean (n=6); bars, SD. p=0.0003 (control group versus 120 mg/kg gefitinib alone group), p=0.0006 (control group versus 60 mg/kg gefitinib and 1 g/kg combine group), and p=0.484 (120 mg/kg gefitinib alone group versus 60 mg/kg gefitinib and 1 g/kg combine group).

FIG. 7 shows the enhancement of antitumor proliferation activity and an induction of apoptosis by composite gefitinib/curcumin treatment using a xenograft tumor model. A, left, Western blot showing that curcumin combined with gefitinib inhibits the expression of EGFR, AKT, c-MET, cyclin D1, PCNA, and iNOS in lung adenocarcinoma tumor tissue; right, Western blot showing that curcumin combined with gefitinib inhibits the expression of procaspase-8, procaspase-3 and procaspase-9 and enhances the amount of full length PARP fragmentation in the lung adenocarcinoma tumor tissues. Data are represented with β-actin used as the internal control. Samples were pooled together from three animals in each group and analyzed; representative results are shown. B, H&E staining, immunohistochemical staining of the proliferation marker PCNA and of apoptosis detection using the TUNEL assay. To do this, s.c. tumor sections from each group were used and the results indicated that curcumin further inhibits tumor cell proliferation and enhances apoptotic cell death induced by gefitinib.

FIG. 8 shows that curcumin attenuates the gastrointestinal adverse effects of gefitinib in vivo. A, left, the animal survival rates for each group indicates that curcumin decreased mouse death caused by the adverse effects of gefitinib; right, photographs of the intestines of mice from each group presented in order to show the curcumin reduced gastrointestinal side effects of gefitinib. B, left, H&E staining of intestine sections from each group shows that curcumin is able to prevent villi damage, which is one of the major adverse effect of gefitinib; right, villi lengths were quantified (bar=200 μm). Columns, mean (n=3); bars, SE. p=0.0001 (120 mg/kg gefitinib alone group versus 120 mg/kg gefitinib plus 1 g/kg curcumin group). C, left, apoptosis detection by TUNEL assay using intestine sections from each group; these indicate that curcumin attenuation of the villi together with cell death caused by gefitinib; right, quantification of TUNEL stained positive cells as described in Materials and Methods. Columns, mean (n=10); bars, SE.

FIG. 9 shows that curcumin attenuates the gastrointestinal adverse effects of gefitinib in vitro. A, Caspase-Glo® 3/7 assay results shows that gefitinib causes the dose-dependent induction of caspase 3/7 activity in IEC-18 cell, but curcumin could reverse this gefitinib-induced caspase activity. Columns, mean (n=3); bars, SD. Data are representative of two independent experiments. B, Western blotting shows that gefitinib increases active-p38 expression in a dose-dependent manner (0-20 μM), but the levels dose not be affected in CL1-5 cell. Data are representative of two independent experiments with β-actin used as the internal control. C, left, Western blotting shows that curcumin and BIRB 796 decrease the gefitinib-induced p38 activation protein level in IEC-18 cell. Data are representative of two independent experiments with β-actin used as the internal control; right, MTT assay shows curcumin and BIRB 796 prevent the IEC-18 cell proliferation from the gefitinib-induced toxic damage. Columns, mean (n=6); bars, SD. Data are representative of two independent experiments.

FIG. 10 shows that curcumin and its analogues LL-17, LL-18, LL-68, and JC-15 (columns 3-7) could attenuate the gefitinib-induced IEC-18 intestinal cells proliferative inhibition. Columns, mean (n=6); bars, SD. *, p<0.05 and ** p<0.01 compare to the gefitinib treatment group (column 2). Data are representative of two independent experiments.

FIG. 11 shows the amino acid sequence of the EFGR protein as in GenBank accession no NM_{—}005228 (SEQ ID NO: 1).

### DETAILED DESCRIPTION OF THE INVENTION

Unless defined otherwise, all technical and scientific terms used herein have the same meaning as commonly understood by a person skilled in the art to which this invention belongs.

All publications mentioned herein are incorporated herein by reference to disclose and describe the methods and/or materials in connection with which the publications are cited.

As used herein, the singular forms “a”, “an”, and “the” include plural referents unless the context clearly dictates otherwise. Thus, for example, reference to “a sample” includes a plurality of such samples and equivalents thereof known to those skilled in the art.

As described above, it is found in the invention that administration of curcumin or its analogue can reduce side effects caused by EGFR-TKI treatment in a patient undergoing the treatment.

Therefore, in one aspect, the present invention provides a method for reducing side effects resulting from treatment using an epidermal growth factor receptor tyrosine kinase inhibitor (EGFR-TKI), comprising administering curcumin or its analogue to a patient undergoing such treatment in an amount effective to reduce the side effects.

Epidermal growth factor receptor (EGFR) is a 170 kilodalton (kDa) membrane-bound protein expressed on the surface of epithelial cells, which is known to involve regulation of cell growth and differentiation and act via regulating the phosphorylation of intrinsic tyrosin kinases. Over expression of EGFR has been reported to occur in various malignant cells and is correlated with a poor prognosis. As used herein, the EGFR protein is disclosed as GenBank accession no NM_{—}005228 (SEQ ID NO: 1).

The term “EGFR-TKI” as used herein refers to an epidermal growth factor receptor tyrosine kinase inhibitor. Certain examples of EGFR-TKIs include gefitinib i.e. N-(3-Chloro-4-fluoro-phenyl)-7-methoxy-6-(3-morpholin-4-ylpropoxy)quinazolin-4-amine (Iressa®) and erlotinib i.e. N-(3-ethynylphenyl)-6,7-bis(2-methoxyethoxy)quinazolin-4-amine (Tarceva®), which are medicines for treating non-small cell lung cancer in clinical.

The term “side effects” as used herein refers to adverse effects induced by EGFR-TKIs such as adverse gastrointestinal effects (e.g. diarrhea, damage on intestine villi/cells, or growth inhibition of intestinal cells) or unfavorable skin conditions (e.g. rash or dry skin). In one embodiment, the side effects are EGFR-TKI induced intestinal cell damage or growth inhibition.

Curcumin (diferuloylmethane) is a highly active component extracted from the plant Curcuma longa, the formula of which (enol form) is as follows:

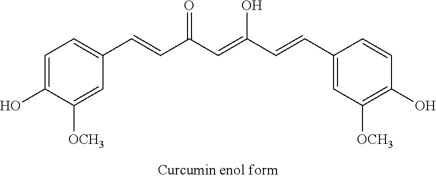

The term “curcumin” as used herein also includes its analogues, derivatives, or salts. A product made from curcumin, such as a food additive or supplement, is also included. In one embodiment, curcumin analogues are selected from the group consisting of LL-17, LL-18, LL-68 and LC-15, the formulae of which are shown below:

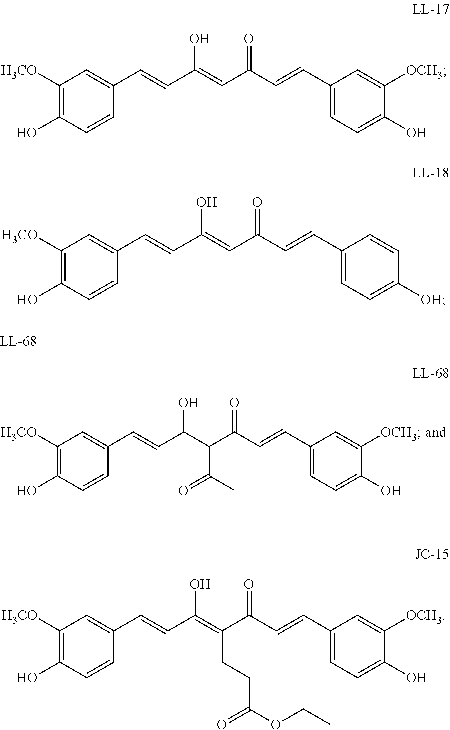

Curcumin or its analogue, derivative or salt thereof as used herein may be synthesized or isolated from natural sources according to common methods known in the art such as those described in Bioorganic & Medicinal Chemistry 2006; 14(8):2527-34; and Journal of Medicinal Chemistry 2006; 49(13):3963-72.

The terms “patient,” “subject” and “individual” are used interchangeably herein and particularly refer to a human subject for which cancer therapy is desired. In one embodiment, the subject is afflicted with non-small-cell lung cancer (NSCLC).

According to the invention, an EGFR-TKI and curcumin or its analogue may be administered in one therapeutic dosage form or in separate therapeutic dosages such as in separate capsules, tablets, containers, or injections. The EGFR-TKI and curcumin or its analogue can be administered simultaneously (concurrently) or sequentially. In one embodiment, the EGFR-TKI and curcumin or its analogue are administered concurrently.

To facilitate delivery, the EGFR-TKI and curcumin according to the invention may be, individually or in combination, formulated into a pharmaceutical composition with a pharmaceutically acceptable carrier. “Pharmaceutically acceptable” as used herein means that the carrier is compatible with the active ingredient contained in the composition, preferably capable of stabilizing the active ingredient, and not deleterious to the subject to be treated. The carrier may serve as a diluent, vehicle, excipient, or medium for the active ingredient. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical composition can additionally include lubricating agents such as talc, magnesium stearate, and mineral oil; wetting agents; emulsifying and suspending agents; preserving agents such as methyl- and propylhydroxy-benzoates; sweetening agents; and flavoring agents.

The pharmaceutical composition according to the invention can be in the form of tablets, pills, powders, lozenges, sachets, cachets, elixirs, suspensions, emulsions, solutions, syrups, soft and hard gelatin capsules, suppositories, sterile injectable solutions, and packaged powders.

The pharmaceutical composition of the invention may be delivered through any physiologically acceptable route such as orally, parentally (e.g. intramuscularly, intravenously, subcutaneously, interperitoneally), transdermally, rectally, by inhalation and the like. In one embodiment, the composition of the invention is orally administrated.

In the invention, it is also found that when an EGFR-TKI is administered to a patient in need thereof in combination with curcumin, the effective amount of EGFR-TKI can be reduced while the therapeutic efficacy is substantially maintained as compared to administering the EGFR-TKI alone.

Therefore, in another aspect, the present invention provides a method for administering an epidermal growth factor receptor tyrosine kinase inhibitor (EGFR-TKI) to a patient in need of a cancer therapy using the EGFR-TKI, comprising administering to the patient a reduced dose of the EGFR-TKI in combination with curcumin or its analogue while efficacy of the EGFR-TKI with respect to the cancer therapy is substantially maintained as compared to that achieved with a standard dose of the EGFR-TKI without administration of the curcumin or its analogue.

An “effective amount” or an “effective dose,” in connection with administration of a pharmacological agent, indicates an amount or dose that results in an intended pharmacological result, such as improvement of symptoms, reduction of side effects, extension of life or improvement of quality of life; in the case of a subject having a malignant tumor, for example, the rate of tumor growth is decreased, the volume of such tumor is reduced, or the tumor is eliminated entirely. The effective amount or dose of a pharmacological agent may vary depending on particular active ingredient employed, the mode of administration, and the age, size, and condition of the subject to be treated. Precise amounts of a pharmacological agent required to be administered depend on the judgment of the practitioner and are peculiar to each individual.

The term “a standard dose” as used herein refers to an effective dose of a therapeutic agent that is recommended by authoritative sources in the pharmaceutical community including the Food and Drug Administration and often used in routine practice. The term “a reduced dose” as used herein refers to a dose that is lower than a standard dose but still retains substantially the same therapeutic effects of the same therapeutic agent. Specifically, according to the invention, a reduced dose of an EGFR-TKI is about 90% or less, 80% or less, 70% or less, 60% or less, 50% or less, of standard therapeutic dose of the EGFR-TKI. In one embodiment of the invention, the reduced dose is about 50% or less of the standard dose of the EGFR-TKI.

It is known that some patients have a poor response to EGFR-TKI treatment and may need a high dose to achieve the required therapeutic effect, which however would cause unacceptable toxicity to the patient and lead to treatment interruption eventually. Surprisingly, the method of the invention is particularly effective in treatment of the patients with resistance to EGFR-TKI. When applied to EGFR-TKI resistant patients, the method of the invention allows a reduced dose of the EGFR-TKI to be administered to the patient while the therapeutic efficacy is substantially maintained as compared to administrating the EGFR-TKI alone. In one embodiment, the patients harbor mutation indicative of resistance to an EGFR-TKI such as T790M substitution in EGFR (SEQ ID NO: 1).

Also provided is a method for treating a cancer patient with resistance to an EGFR-TKI, which comprises jointly administering to said patient an effective amount of the EGFR-TKI with curcumin or its analogue.

The term “treating” as used herein refers to the application or administration of a composition including one or more active agents to a subject, who has a disease particularly to be treated by EGFR-TKI, including but are not limited to, tumor or cancer, a symptom of the disease, or a predisposition toward the disease, with the purpose to cure, heal, alleviate, relieve, alter, remedy, ameliorate, improve, or affect the disease, the symptoms of the disease, or the predisposition toward the disease.

The present invention will now be described more specifically with reference to the following embodiments, which are provided for the purpose of demonstration rather than limitation.

Materials and Methods

1. Reagents

Curcumin (purity by HPLC: 98.0%) for the in vitro studies was purchased from Calbiochem (Darmstadt, Germany). In vivo, the curcumin (purity˜70%) was from Sigma (St Louis, Mo.). Gefitinib (Iressa®), ZD1839, was kindly provided by Astra-Zeneca Pharmaceuticals (Macclesfield, UK). Stock solutions for curcumin and gefitinib were prepared in dimethyl sulfoxide (DMSO) and stored at −20° C. The compounds were diluted in fresh media before each experiment, and the final DMSO concentration was lower than 0.1%. Curcumin and gefitinib for the animals was prepared by fully suspending the drug in propylene glycol (J. T. Baker, Phillipsburg USA).

2. Cell Lines and Culture Conditions

The human lung adenocarcinoma cell line with highly invasive capacities (CL1-5) was established previously (American Journal of Respiratory Cell & Molecular Biology 1997; 17(3):353-60). The human lung carcinoma cell lines A549, H1299, H1650, and H1975 were obtained from American Type Culture Collection (Manassas, Va.). PC-9 was a kindly gift from Dr. Chih-Hsin Yang (National Taiwan University Hospital, Taiwan). These cells were grown in RPMI 1640 medium (Life Technologies Rockville, Md.). The IEC-18 rat intestinal epithelial cell line (BCRC 60230) was grown in DMEM medium (Life Technologies Rockville, Md.) supplemented with 10% fetal bovine serum (FBS) (Life Technologies). The media for each of the above contained penicillin and streptomycin (100 mg/ml each) and the cell lines were incubated at 37° C. in a humidified atmosphere with 5% CO₂.

3. Proliferation Assay

A MTT [3-(4,5-dimethylthiazolyl-2)-2,5-diphenyltetrazolium bromide] (Sigma, St Louis, Mo.) assay was performed to determine cell proliferation. Briefly, CL1-5, A549, PC-9, H1650, H1975 and IEC-18 cells were plated in 96-well plates at a density of 5×10³ cells/well. After incubating for 24 h, cells were then treated with different concentrations of curcumin and/or gefitinib for 72 h. In addition, the IEC-18 cells were treated with curcumin, BIRB 796 and/or gefitinib for 24 h incubation. MTT solution to a final concentration in the culture medium 0.5 mg/ml was then added to the wells. After a further 1.5 h of incubation, the medium was removed and DMSO was added to the plates. The color intensity of the solubilized formazan was measured at 570 nm using a multi-label plate reader (Vector3; Perkin-Elmer, USA).

4. Colony Formation Assay

CL1-5, A549 and H1975 cells were plated in 6-well plates (100 cells per well) with culture medium. After incubating for 24 hours, the cells were treated with gefitinib or curcumin alone or with a combined treatment as indicated. The cells were cultured with the agents for 5 days and then the medium completely changed; the cells were then incubated for a further 9 days. Colonies were then stained using 0.001% crystal violate and the number of colonies per well counted.

5. Western Blot Analysis

Western blotting was used to determine the protein expression levels of EGFR, pEGFR, Akt, pAkt, cyclin D1, PCNA, iNOS and various apoptosis related proteins (pro-caspase-3, 8, 9 and PARP). Cells were plated in 10-cm dish at a density of 1×10⁶. After incubating overnight, the cells were serum starved for 24 h in medium with no FBS. Next the cells were treated with different concentrations of curcumin and/or gefitinib for 1 h under the serum-free conditions and then were stimulated with 20 ng/ml, EGF for 30 min. The IEC-18 cell was plated in 10-cm dish at a density of 1×10⁶. After incubating overnight, the cells were treated with different concentrations of gefitinib and/or curcumin or BIRB 796 for further 24 h. These cells were washed three times with ice-cold PBS and their protein extracted. In addition, CL1-5 tumor tissue (100 mg) was harvest from each of the group and then minced in lysate buffer. The protein extracts were obtained using mammalian protein extraction reagent (Pierce, Rockford), which contains a protease inhibitor and a phosphatase inhibitor (Sigma, USA). SDS/PAGE using a 10% resolving gel was carried out to separate the proteins (25 mg/lane). Antibodies against phospho-EGFR (Tyr1068), phospho-Akt (Ser473), Akt, c-MET, caspase-3, caspase-8, caspase-9, PARP, active-p38 and p38 were purchased from Cell Signaling Technology (Beverly, Mass.). Antibodies against both forms of EGFR, cyclin D1, PCNA, and iNOS were purchased from Santa Cruz Biotechnology (Santa Cruz, Calif.). The antibodies were used according to the conditions recommended by the manufacturer. Bound antibody was detected using the Enhanced Chemiluminescence System (Santa Cruz, Calif.). Chemiluminescent signals were captured using the Fujifilm LAS 3000 system (Fujifilm, Tokyo, Japan). All experiments were performed at least three times in duplicate.

6. Flow Cytometry

Cells were seeded in 6-mm culture plate at the density of 10⁵ cells per dish. After incubating for 24 h, the cells were serum starved overnight. The cells were then treated with curcumin and/or gefitinib for further 72 h. Adherent and floating cells were collected separately and resuspended in cold 1×PBS for further analysis. The cells were stained with an Annexin V-FITC Apoptosis Kit (BD Pharmingen, USA) to monitor apoptosis cells and propidium iodide (PI) to detect dead cells. The samples were analyzed on a FC 500 Flow Cytometry Systems (Beckman Coulter). Unstained cells were classified as “live”, cells stained for annexin V only were classified as “early apoptotic”, cells stained for both annexin V and PI were classified as “late apoptotic” and cells stained for PI only were classified as “dead”.

7. Real-Time Quantitative RT-PCR

The expression level of EGFR was detected by real-time PCR on an ABI prism 7900 sequence detection system (Applied Biosystems). The EGFR primers used were as follows: forward primer EGFR-F, 5′-GTGACCGTTTGGGAGTTGATGA-3′; and reverse primer EGFR-R, 5′-GGCTGAGGGAGGCGTTCTC-3′. The TATA-box binding protein (TBP) was used as the internal control (GenBank X54993). The primers and probe used for the quantitative RT-PCR of TBP mRNA were as described previously (24, 25). The relative expression level of EGFR compared with that of TBP was defined as −ΔCT=−[CT_{EGFR}−CT_{TBP}]. The EGFR mRNA/TBP mRNA ratio was calculated as 2^{-ΔCT}×K, in which K is a constant. All experiments were performed three times in triplicate.

8. In Vivo Study Protocol

We perform the in vivo study in mice according to the protocols approved by the National Yang-Ming University Animal Care and Use Committee. The CL1-5 cells were calculate in terms of cell survival and cell number using trypan blue and then 1×10⁶ live CL1-5 cells in 100 μl HBSS were injected subcutaneously into 6-weeks-old SCID mice (supplied by the animal center in the College of Medicine, National Taiwan University, Taipei, Taiwan). To examine whether curcumin can enhance the antitumor effects of gefitinib, the mice were randomized into five groups (n=9) at 1 week after cell injection; these were: (1) vehicle control; (2) curcumin alone (1 g/kg); (3) gefitinib alone (120 mg/kg); (4) 60 mg/kg gefitinib plus 1 g/kg curcumin; and (5) 120 mg/kg gefitinib plus 1 g/kg curcumin. Curcumin and gefitinib were fed to the animals by oral administration once daily at indicated treatment dose. Tumor sizes were monitored every 4 days by electronic vernier caliper and the tumor volume was calculated using the formula V=0.4×ab², where a and b are the longest and shortest diameters of the tumors, respectively. After 3 weeks, the mice were sacrificed, the subcutaneous tumors were excised; they were then frozen in liquid nitrogen and finally stored at −80° C. Intestine samples from the mice in each group were fixed using Bouin's fluid and paraffin embedded for routine hematoxylin and eosin staining.

9. Immunohistochemical Staining for PCNA, and the Cell Apoptosis Detection Assay

Cell proliferation analysis was performed on the paraffin-embedded tumor tissue samples using PCNA staining. Briefly, a rabbit anti-human PCNA polyclonal antibody (Santa Cruz, Calif.) was used in the primary reaction. The DAKO EnVision System, containing a secondary horseradish peroxidase-conjugated anti-mouse antibody complex, was used with 3,3′-diaminobenzidine to detect the PCNA.

Colorimetric immunohistochemical staining for apoptotic cell death (TUNEL) was performed on the paraffin-embedded tumor and intestine tissue sections using the In Situ Cell Death Detection Kit, POD (Roche Diagnostics, Germany); the sections were also counterstained with Gill's hematoxylin. TUNEL-positive cells were examined in 10 random fields from three intestines of each of the treatment groups and then expressed as the mean number of TUNEL-positive cells ±SE per high-power field (×400 magnification).

10. Measurement of Caspase Activity

Caspase activity was detected by using Caspase-Glo® 3/7 assay kit (Promega Corporation, Australia). Briefly, The IEC-18 cell was seeded in 96-well white luminometer assay plates at a density of 1×10⁴ cells per well and incubated at 37° C. After incubating for 24 h, cells were then treated with different concentrations of gefitinib and/or curcumin for further 24 h. 100 μl caspase 3/7 reagents were added to each well and incubated for 1 h on rotary shaker at room temperature. The luminescence intensity for each well was measured using a multi-label plate reader (Vector3; Perkin-Elmer, USA).

11. Statistical Analysis

All experiments were performed in triplicate and analyzed by ANOVA (Excel, Microsoft; Taipei, Taiwan). Comparisons were made using a two-tailed Student's t test and significant differences were defined as p<0.05. Where appropriate, the data are presented as the mean±SD.

Results

1. Curcumin can Inhibit Lung Adenocarcinoma Cell Proliferation, EGFR and AKT Protein Expression and Phosphorylation

To develop new agents or compounds for enhancing the anti-tumor effects, reducing the dosage, or overcoming the resistance of gefitinib in NSCLC patients, a high-throughput drug screening system with different gefitinib resistant cell lines was applied for screening hundreds of compounds from herbs in our laboratory. Table 1 shows the EGFR status and ethnicities of the NSCLC cell lines used herein.

| Cell line | EGFR status | Ethnicity |
|---|---|---|
| CL1-5 | wild-type EGFR | Asian |
| A549 | wild-type EGFR | Caucasian |
| H1299 | with wild-type EGFR but p53-null | Caucasian |
| H1650 | in-frame deletions (ΔE746-A750) of exon 19 in | Caucasian |
| | EGFR, but loss of PTEN protein | |
| H1975 | L858R substitution in exon 21 and secondary | Caucasian |
| | T790M substitution in exon 20 in EGFR | |
| PC-9 | in-frame deletions (ΔE746-A750) of exon 19 in | Asian |
| | EGFR | |

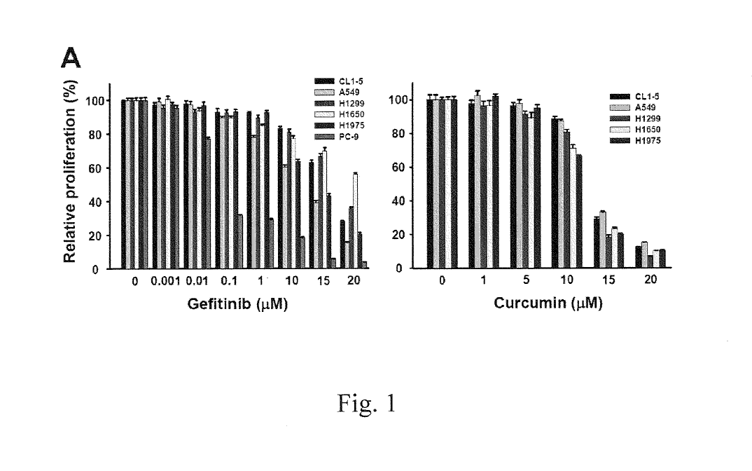

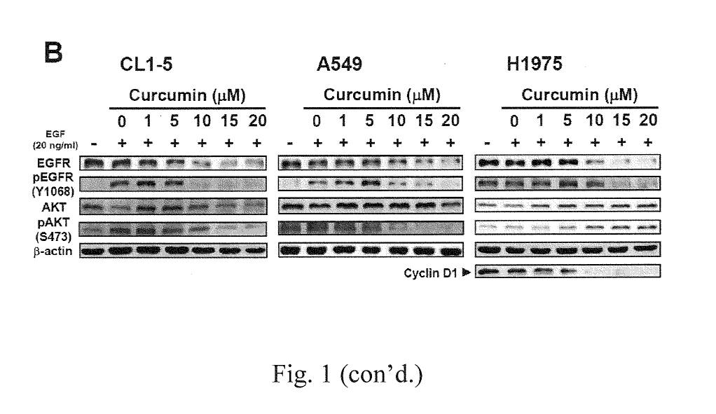

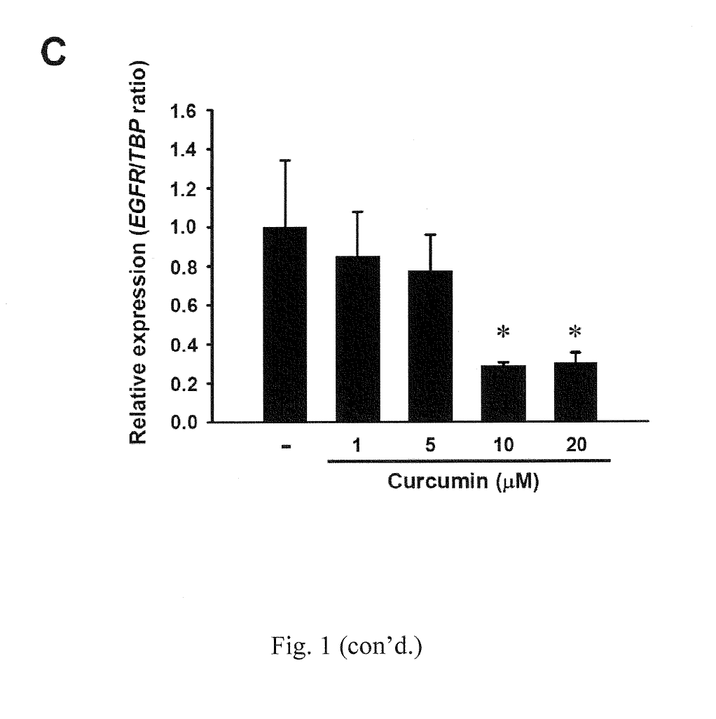

Curcumin was selected as a potential candidate. We confirmed that curcumin exhibited significantly inhibitory effects on cell proliferation in the gefitinib resistant NSCLC cell lines, including CL1-5 (wt-EGFR), A549 (wt-EGFR), H1299 (wt-EGFR), H1650 (in-frame deletions ΔE746-A750 of exon 19 in EGFR with PTEN loss), and H1975 (L858R and T790M mutations in EGFR). As shown in FIG. 1A, compared with the gefitinib-sensitive cell lines, PC-9 (IC₅₀<0.1 μM), the CL1-5, A549, H1299, H1650, and H1975 showed an overall pattern of increased resistance (IC₅₀>10 μM) when incubated with gefitinib for 72 hours (FIG. 1A Left); nonetheless, the proliferation of these gefitinib-resistant cell lines was inhibited by curcumin in a concentration-dependent manner (FIG. 1A right).

The EGFR signaling pathway is known to be highly correlated with tumor progression and therefore the effect of curcumin on the expression level and activity (phosphorylation) of EGFR and AKT in CL1-5, A549 and H1975 cells was examined (FIG. 1B). The results indicated that curcumin is able to reduce EGFR, pEGFR, AKT, and pAKT protein expression in both CL1-5 and A549 cells in a concentration dependent manner (FIG. 1B). Even though AKT phosphorylation was not altered by curcumin in the H1975 cells, an EGFR down-stream signaling factor, namely the level of cyclin D1, was still significantly reduced by curcumin (FIG. 1B, right). Furthermore, the RT Q-PCR results showed that curcumin is able to reduce EGFR mRNA expression in a concentration-dependent manner (FIG. 1C). These results indicated that curcumin does exhibit a potential anticancer effect by diminishing EGFR signaling in gefitinib-resistant NSCLC cells.

2. Binding Activity of Curcumin on EGFR

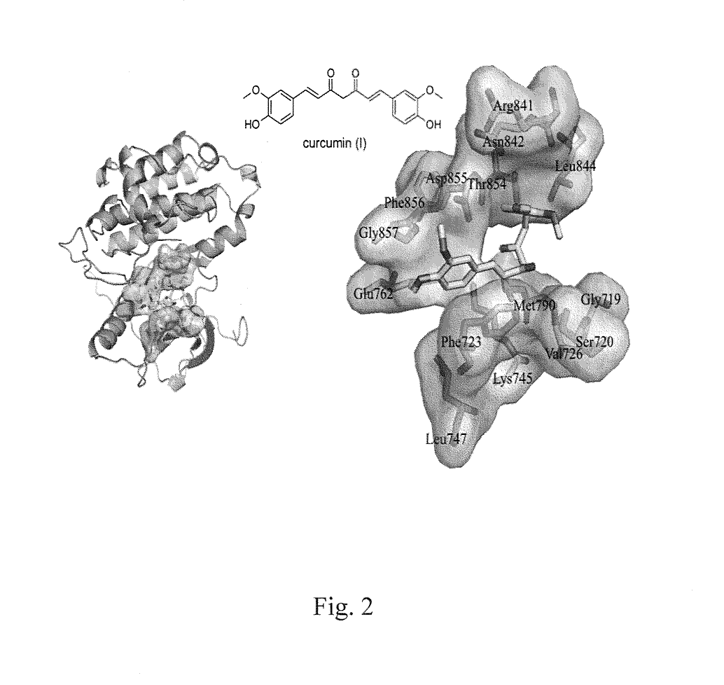

We investigated the binding activity of curcumin on EGFR and compared it with that of gefitinib. The results show that predicted 3-D conformation of curcumin have relatively high score bound to the open-form wild-type EGFR protein by LIBDOCK (curcumin's score=89.5; gefitinib's score=80.3). In addition, FIG. 2 shows the binding of curcumin on the tyrosine phosphorylation site of EGFR with T790M resistant mutation. Take together, these results showed that curcumin exhibits higher binding activity on EGFR when compared with gefitinib, and T790M resistant mutation would not affect the binding activity of curcumin on EGFR.

3. Curcumin Accelerates EGFR Degradation and Downregulates EGFR Protein Level

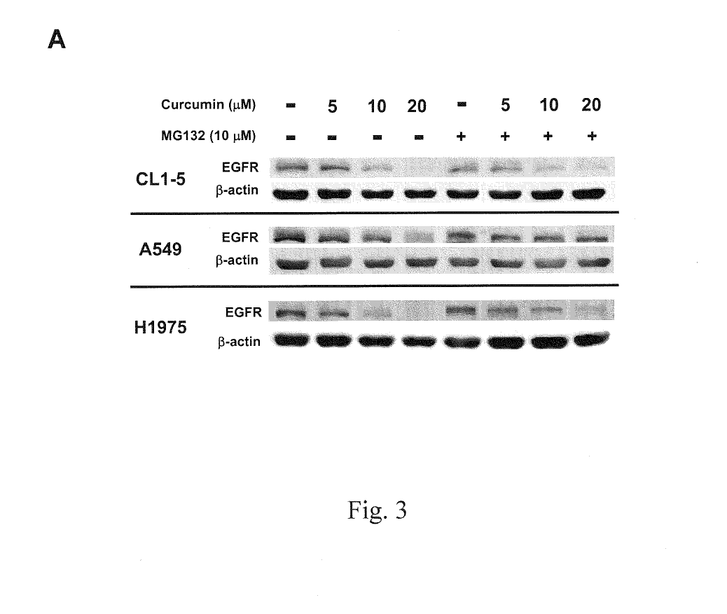

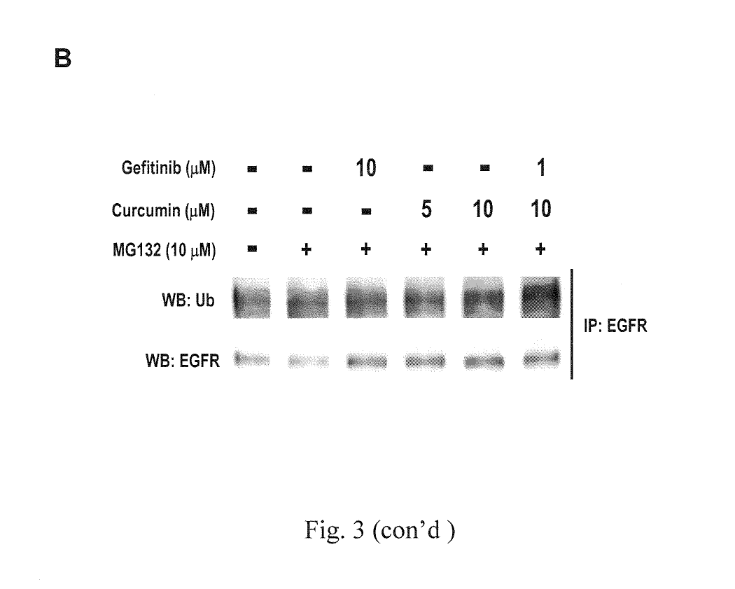

We examined whether curcumin was able to accelerate EGFR degradation in the translational level. Lung adenocarcinoma cells were pre-treated with or without MG132 for 3 h and then treated with curcumin as indicated. Our data showed that curcumin was able to inhibit the EGFR protein expression in a dose-dependent manner; however, while the cells were pre-treated with MG132, a proteasome inhibitor, the EGFR protein level can be recovered (FIG. 3A). These results might indicate that curcumin was able to decrease EGFR protein expression level through accelerating ubiquitin-proteasome ability.

We also found that EGFR protein level in the gefitinib combined with curcumin group were lower than curcumin alone. Thus, we processed the immunoprecipitation assay to examine the observations. The results showed that gefitinib (10 μM) didn't alter the EGFR ubiquitin level, however, in the combine treatment group, gefitinib (1 μM) and curcumin (10 μM) was able to increase the EGFR protein ubiquitination compare to the other single treatment group (FIG. 3B). This data indicated that curcumin can enhance the gefitinib anti-tumor property by downregulating EGFR protein level.

4. Combining Curcumin with Gefitinib is Able to Improve the Anti-Tumor Effects of Gefitinib in NSCLC Cells with Either a Wild-Type or Mutant EGFR

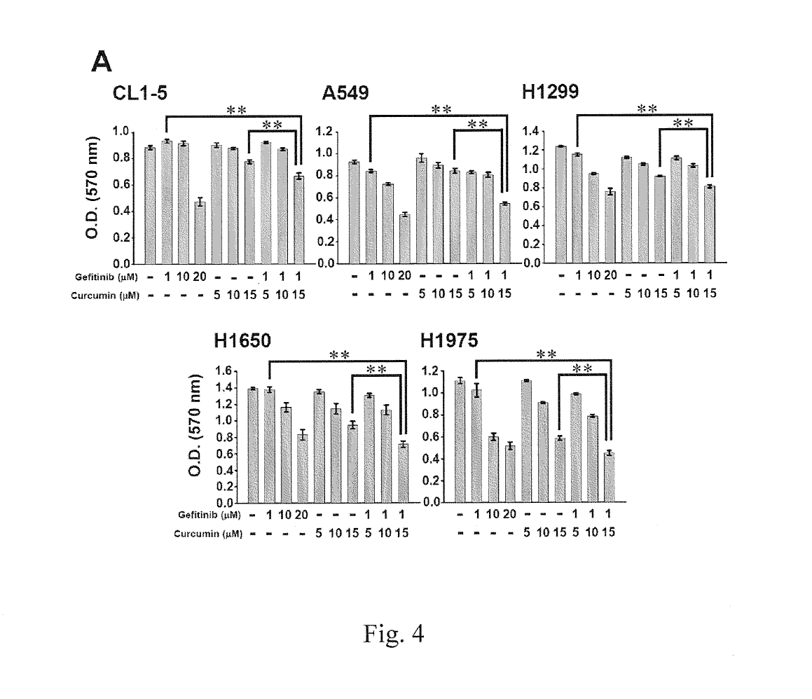

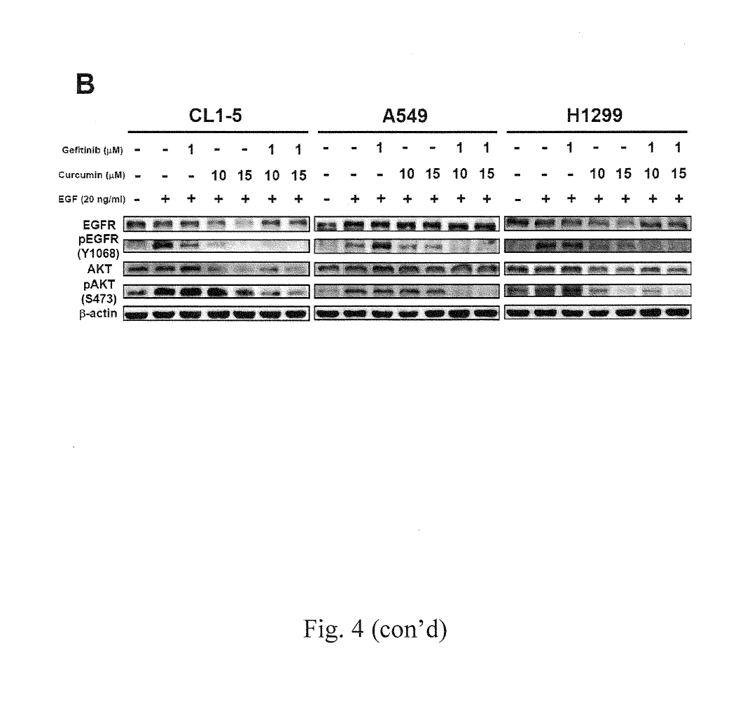

The CL1-5, A549, H1299, H1650 and H1975 cell lines, with either a wild-type or mutant EGFR, were used to evaluate whether curcumin can increase the antitumor effects of gefitinib in various gefitinib-resistant NSCLC cells that differ in their EGFR status. The cell proliferation assay showed that gefitinib (≦10 μM) or curcumin (≦15 μM) treatment alone only produced a slight inhibition of cell proliferation (FIG. 4A). However, when curcumin (15 μM) was combined with gefitinib (1 μM) there was a significant reduction in cell proliferation with all five of the gefitinib-resistant NSCLC cell lines and the anti-proliferation effect was equivalent to treatment with a high dose of gefitinib (20 μM) (FIG. 4A). EGFR signaling was also found to be inhibited significantly by the combined curcumin and gefitinib treatment when CL1-5, A549 and H1299 cells were examined in terms of the expression levels of EGFR, pEGFR, AKT, and pAKT. These results indicate that gefitinib (1 μM) or curcumin (10 and 15 μM) alone has only a minimal suppressive effect on pEGFR and pAKT (FIG. 4B), whereas, the combination of gefitinib (1 μM) and curcumin (15 μM) produced a significant blockade of EGFR and AKT phosphorylation (FIG. 4B). These results suggest that curcumin may be able to enhance the anticancer effects of gefitinib when this drug is used to treat gefitinib-resistant cancer cells.

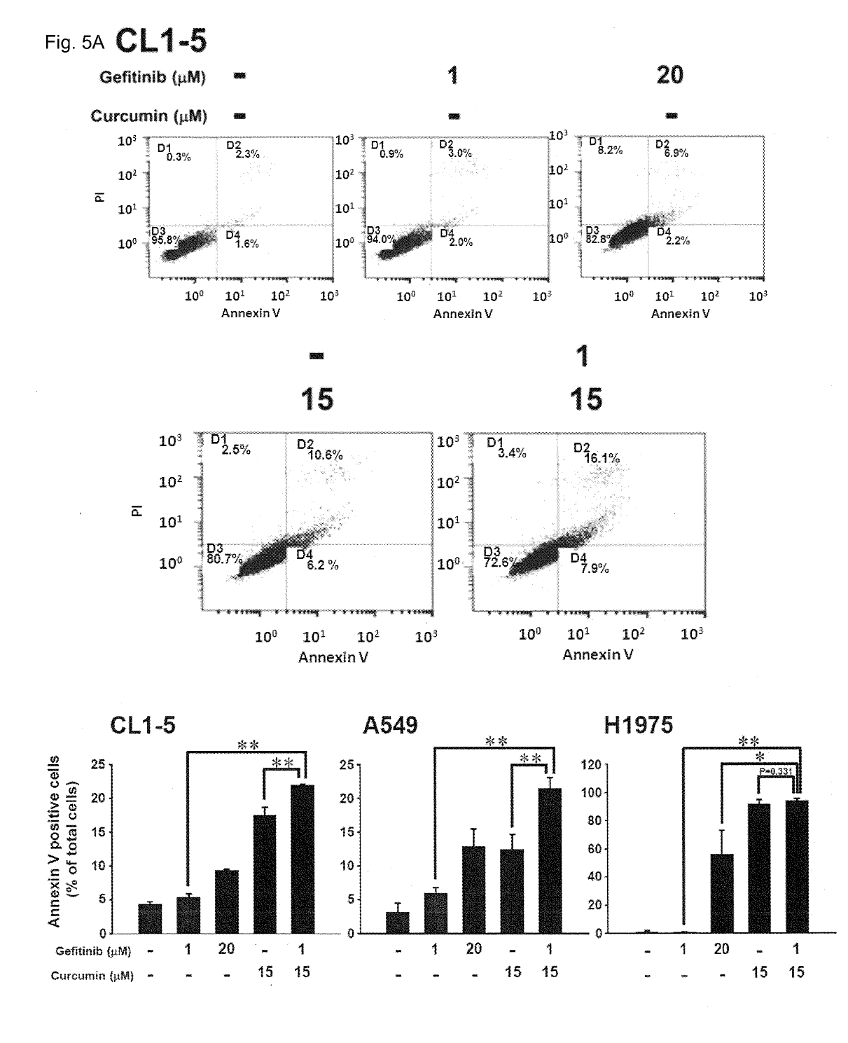

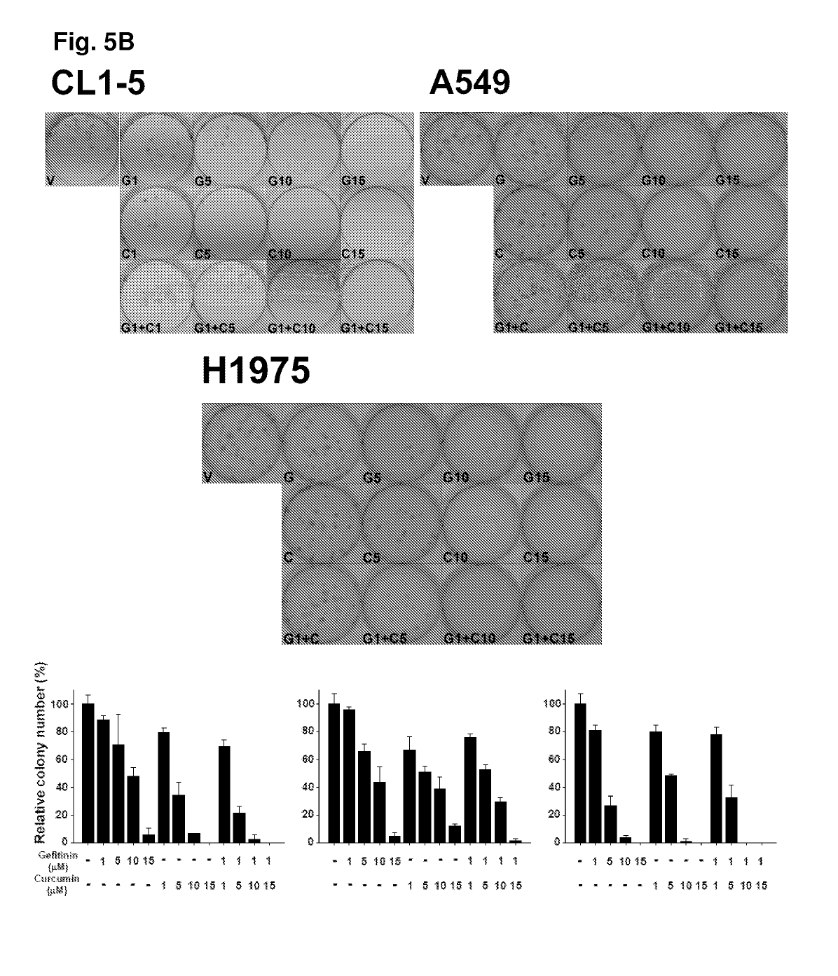

In addition, we investigated whether curcumin is able to increase the amount of apoptosis caused by gefitinib in the CL1-5, A549 and H1975 cell lines. A flow cytometry assay using propidium iodide/annexin-V, staining indicated that gefitinib (1 μM) combined with curcumin (15 μM) induced a higher level of apoptosis than a high concentration of gefitinib alone (20 μM) in CL1-5 and A549 cells (FIG. 5A, left and meddle). Curcumin and gefitinib together also elevated the number of annexin V positive cells compared to gefitinib alone in H1975 cells, but this level was similar to curcumin (15 μM) alone; this lack of induction by the two drugs together can be explained by the fact that 15 μM curcumin with or without gefitinib is able to induce apoptosis in about 90% of H1975 cells (FIG. 5A, right). Taken as a whole, these results seem to indicate that curcumin is able to enhance gefitinib-induced cell apoptosis in gefitinib-resistant cells.

As the next step, we examined whether curcumin was able to enhance the anti-tumorigenicity of gefitinib in CL1-5, A549 and H1975 cells using a colony formation assay. The results were similar to the MTT assay and it was found that a combination of curcumin and gefitinib significantly inhibited colony formation by CL1-5, A549 and H1975 compared to the drug-free control and either drug treatment alone (FIG. 5B). The above in vitro studies support the hypothesis that curcumin is able to enhance the anticancer activities of gefitinib when this drug is used to treat gefitinib-resistant lung adenocarcinoma cells; this effect seems to be independent of the presence of mutation in EGFR mutation or any other genetic alteration.

5. Curcumin Enhances the Antitumor Properties of Gefitinib in Human Lung Adenocarcinoma Cell Xenografts In Vivo.

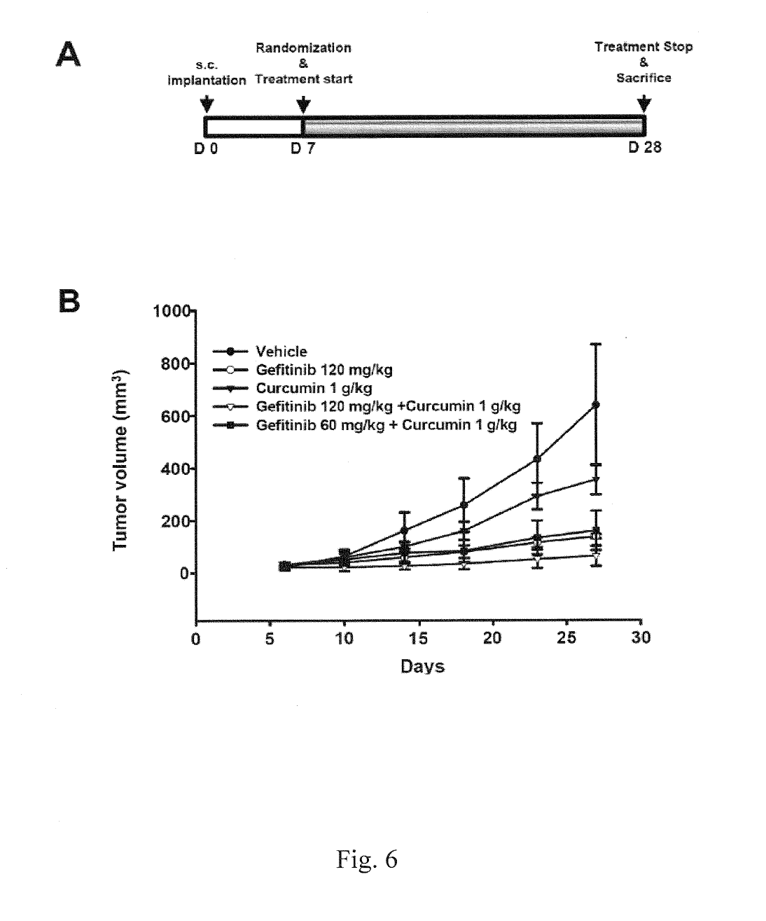

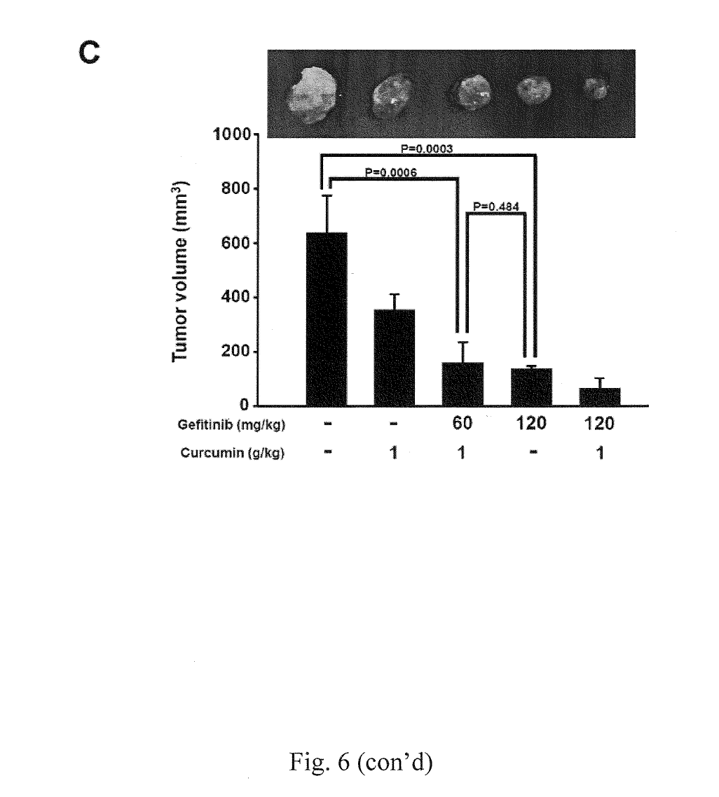

The next step was to investigate whether curcumin is able to enhance the antitumor activity of gefitinib in vivo. To do this, CL1-5 cells were transplanted subcutaneously into SCID mice. After one week, when the tumors were palpable (3-5 mm), the mice were randomized into five groups (FIG. 6A). These five groups were then subjected to different treatment regimes, these were vehicle only as a control, curcumin (1 g/kg), gefitinib (120 mg/kg), curcumin (1 g/kg) combined with a low dose gefitinib (60 mg/kg), and curcumin (1 g/kg) combined with a high dose of gefitinib (120 mg/kg). These treatments were given orally once each day and the mice were sacrificed after 4 weeks. The tumor volumes of each of the groups were monitored every 4 days over this period (FIG. 6B). The tumor size of the control animals averaged 638.55 mm³ at the end of the study. Significantly, the tumor size of the curcumin plus high dose gefitinib combination treatment group showed a major reduction to only 64.15 mm³ (p=0.0001 versus control), while the curcumin plus low dose gefitinib also showed a reduction to an average of 160.59 mm³ (p=0.0006 versus control). The average tumor sizes of the curcumin alone group and the gefitinib alone group were 354.91 (p=0.015 versus control) and 138.32 mm³ (p=0.0003 versus control), respectively. The antitumor activity of curcumin in combination with gefitinib thus shows an enhance effect compared to the curcumin and gefitinib alone groups (p<0.01 versus curcumin and p=0.015 versus gefitinib). In addition, the group treated with curcumin and a low dose of (60 mg/kg) showed a similar level of antitumor activity to that of the 120 mg/kg gefitinib alone (p=0.483) (FIG. 6C). Thus, combining curcumin with a low dose of gefitinib might save half the dose of gefitinib and obtain the same inhibition of tumor progression. These results clearly show that curcumin enhances the antitumor activity of gefitinib.

6. Curcumin Enhances the In Vivo Antitumor Effect of Gefitinib by Reducing EGFR-Related Signaling and Affecting the Regulation of Apoptosis

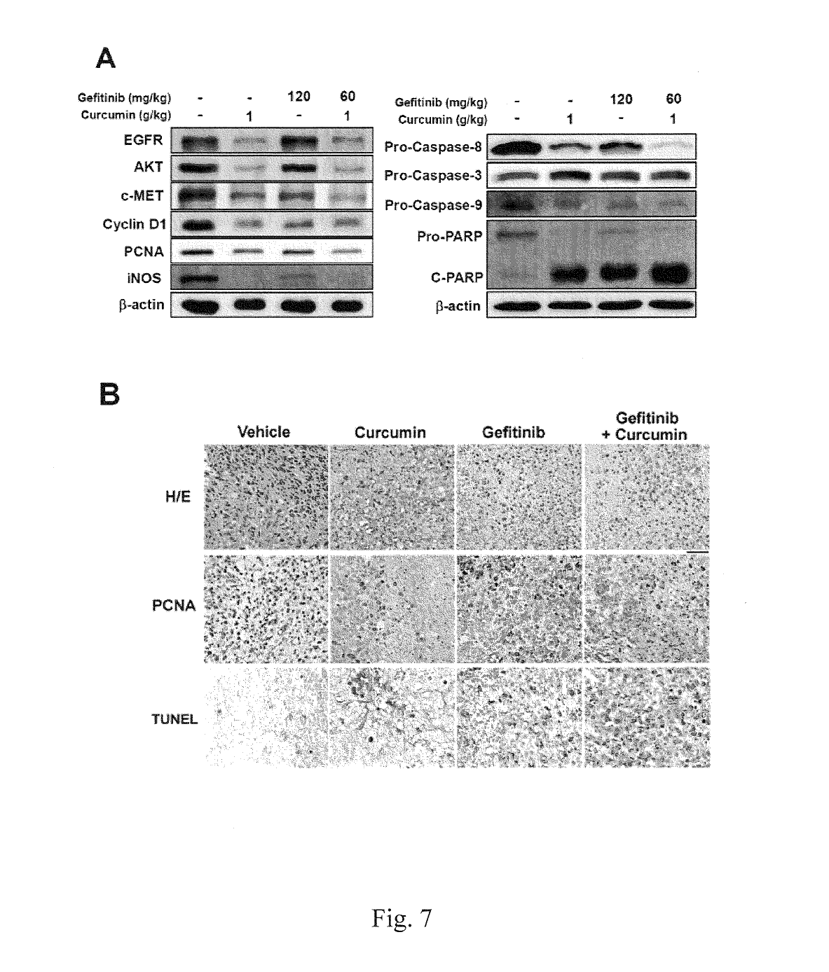

In order to investigate the molecular mechanisms involved in the antitumor activity of combined curcumin and gefitinib treated mice, the protein lysates from the various tumor tissues were analyzed by Western blot analysis; this approach was used to measure the protein levels of EGFR, AKT, cyclin D1, c-MET, PCNA, and iNOS in the tumors. FIG. 7A showed that the levels of EGFR and AKT were decreased in the tumors of SCID mice after treatment with either curcumin alone or curcumin and gefitinib. In addition, the protein levels of cyclin D1, c-MET, PCNA, and iNOS were also shown to be significantly altered in these tumors (FIG. 7A, left). To confirm the apoptosis-induction effects of curcumin combined with gefitinib in vivo, apoptosis-related signaling was investigated, including caspases and PARP. The results showed that curcumin is able to enhance the activity levels of caspase-3, caspae-8, caspase-9 and PARP, especially in the curcumin plus gefitinib group (FIG. 7A, right). These results indicated that curcumin would seem to be potentiating the anticancer activity of gefitinib in the resistance cancer cells in vivo by decreasing EGFR signaling, c-MET, PCNA and iNOS, and by upregulating the apoptosis pathways.

We next examined the proliferation marker PCNA using immunohistochemical staining and cell death by the TUNEL assay; this was done in paraffin-embedded tumor tissue samples. The results in FIG. 7B showed that the PCNA marker in the curcumin treated samples and in the curcumin combined with gefitinib samples was decreased in a manner similar to that of PCNA protein expression (FIG. 7A, left). In addition, the in situ cell death detection assay indicated that treatment with curcumin combined with gefitinib significantly increased cell apoptotic activities compared to the control and to the gefitinib only groups (FIG. 7B). These results also showed that curcumin alone induced cell apoptosis when compared to the control (FIG. 7A, right). These results confirm the anti-proliferation and apoptosis inducing abilities of curcumin in vivo.

7. Curcumin Attenuates the Adverse Gastrointestinal Effects of Gefitinib

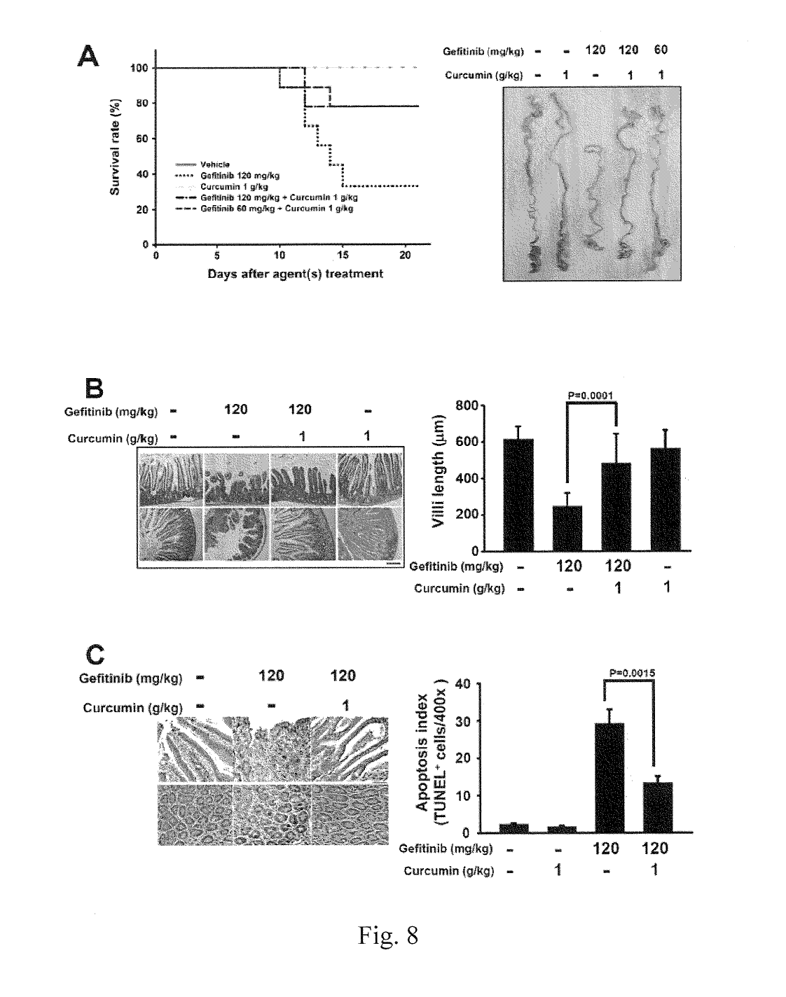

During the in vivo xenograft study, the gefitinib-treated mice showed dramatic body weight lost (data not shown) and there was also an obviously diarrhea side effect; this is similar to previous reports in clinical literature and was even severe enough to result in death within the group. Interestingly, when gefitinib was combined with curcumin, it was able to prevent the body weight loss and it also significantly reduced number of deaths among the mice (the survival rate with the combined therapy was 78% compared to 33% for the gefitinib therapy) (FIG. 8A, left). The morphology and histology of the intestine was then examined to investigate the effect of curcumin as it attenuates the gastrointestinal adverse effects of gefitinib. The results showed that the full length of the intestine in the gefitinib group was shorter and thinner than in the combined therapy group (FIG. 8A, right). There were no obvious differences between the control and curcumin groups or between the control and curcumin plus gefitinib groups. Furthermore, the length of intestine villi obtained from the curcumin and gefitinib combined group were longer and had greater integrity compared to gefitinib-treated group (p=0.0001) when examined by H&E stain (FIG. 8B). Furthermore, curcumin combined with gefitinib was able to significant reduce apoptosis in the villi compared to the gefitinib only group (p=0.0015) when this was assessed by TUNEL assay in vivo (FIG. 8C). Therefore, it would seem that curcumin is able to improve the survival rate of mice treated with gefitinib and can also reduce the GI adverse effects of gefitinib.

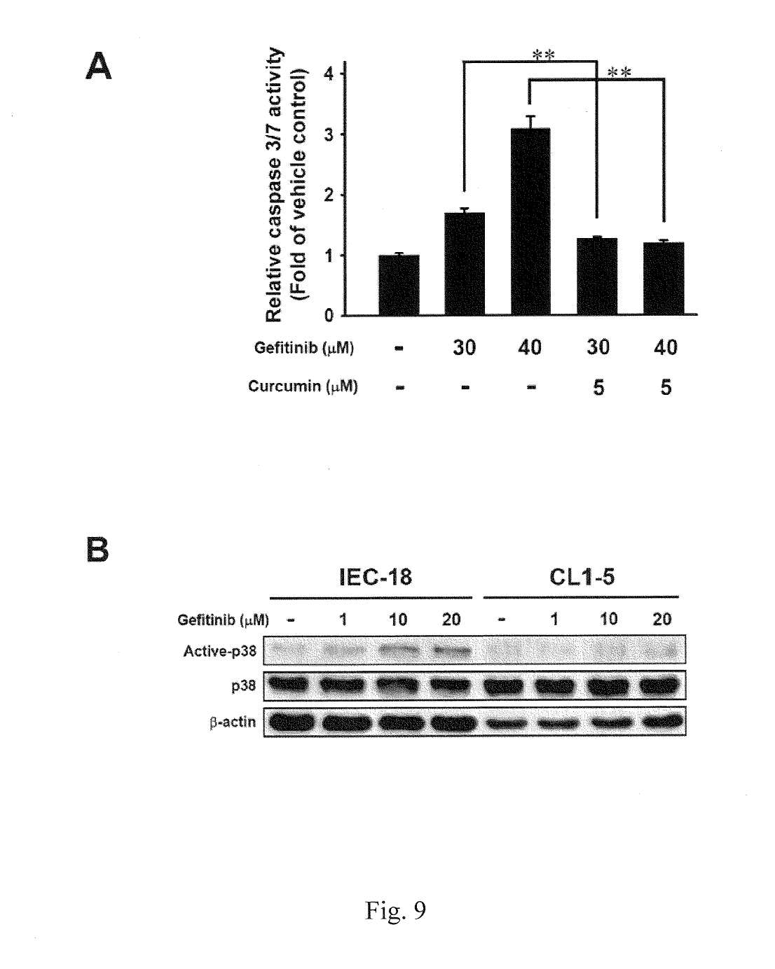

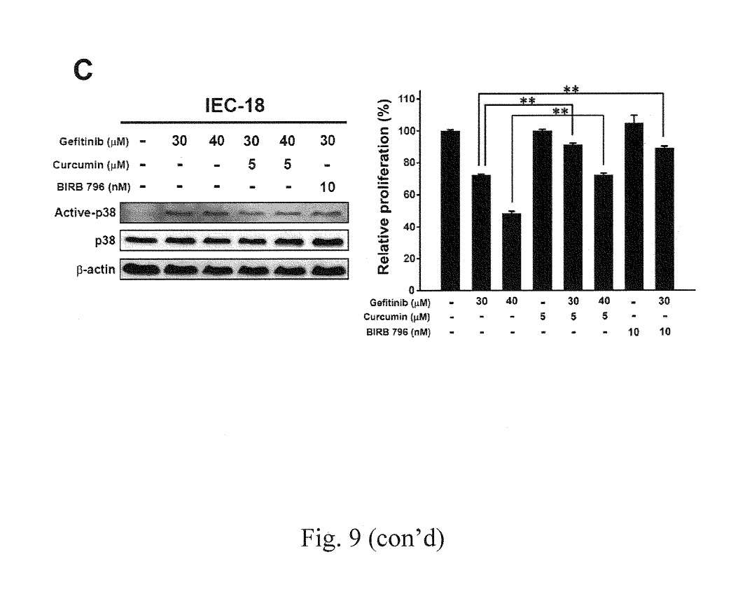

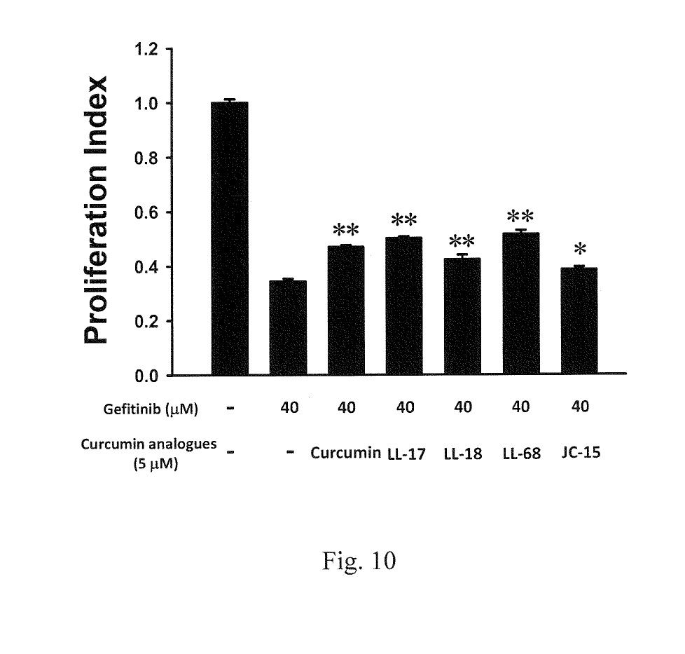

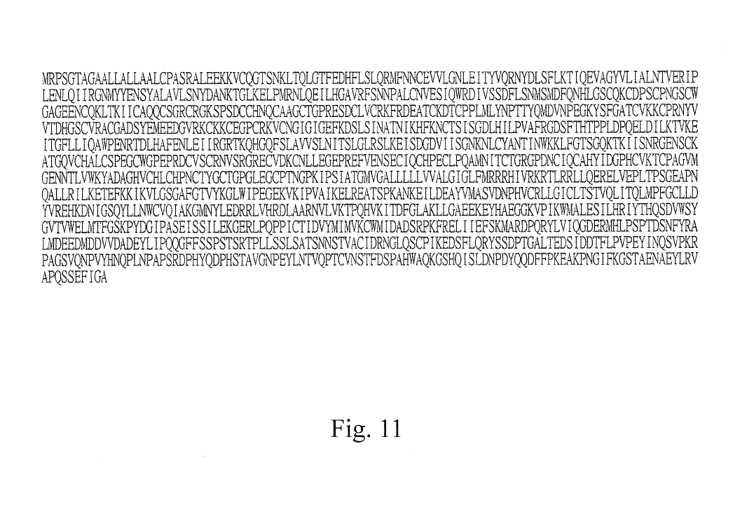

Finally, we investigated the protective effect of curcumin on gefitinib-induced intestinal epithelial cell apoptosis in vitro using the non-transformed intestinal epithelial cell line IEC-18. The Caspase-Glo® 3/7 assay showed that gefitinib (IC₂₅ at 30 μM and IC₅₀ at 40 μM of gefitinib for IEC-18 cell, respectively) was able to induce caspase 3/7 activities in IEC-18 cell, whereas, 5 μM curcumin (non-toxic dosage) could significantly inhibit the gefitinib-induced caspase 3/7 activities (FIG. 9A). The result showed that curcumin is able to prevent the intestinal epithelial cell from gefitinib-induced apoptosis; this effect is obviously similar to the previous observation in vivo. We next to determine which one of possible mechanisms involve in this protective effect of curcumin on gefitinib-induced cell apoptosis. Previous reports has indicated that gefitinib can induce apoptosis in intestinal epithelial cells via p38 mitogen-activated protein kinase (MAPK)-dependent activation (Gastrointestinal & Liver Physiology 2007; 293(3):G599-606), which might be the possible mechanisms for gefitinib-related adverse effects in the GI. Herein, we found that the active-p38 significant increased in a dose dependent manner of gefitinib (0-20 μM) treatment in IEC-18 cell, but the CL1-5 cell did not elevate the p38 activation at the same culture conditions (FIG. 9B). In addition, the gefitinib-induced p38 MAPK activation was significantly inhibited by curcumin in IEC-18 cell: the results indicated that curcumin (5 μM) and the selective p38 MAPK inhibitor BIRB 796 (10 nM) were able to significantly decrease the active-p38 expression on gefitinib-treated IEC-18 cells (FIG. 9C, left). Furthermore, the MIT assay showed that gefitinib (30 and 40 μM) was able to significantly reduce IEC-18 cell survival, whereas, curcumin (5 μM), as well as the BIRB 796 (10 nM), could rescue the cells from this toxic effect of gefitinib, significantly (FIG. 9C, right). Curcumin analogues, LL-17, LL-18, LL68 and JC-15, were also tested and found to have the protective effect to reduce gefitinib-induced intestinal cell death (FIG. 10). Take together; these results showed that curcumin can attenuate the gefitinib-induced complications in the intestine.

In conclusion, our results showed the prominent activity of curcumin as an enhancer of gefitinib's anti-tumor abilities. The agent also attenuates the diarrheal side effects of gefitinib and thus may be a good adjuvant for lung cancer patients. Clinically, the price of lung cancer targeted therapy is quite high and adverse effects are always the critical issues during treatment. Curcumin is a common and cheaper agent and it seems to be able to enhance the effectiveness of gefitinib and thus reduce the costs related to the medical and patient financial burden. To reduce the dosage of gefitinib, cut-down the costs, and prevent the side effects, we suggest that curcumin should be a good adjuvant for NSCLC cancer patients during gefitinib treatment.

I/We claim:

1. A method for reducing side effects resulting from treatment using an epidermal growth factor receptor tyrosine kinase inhibitor (EGFR-TKI), comprising administering curcumin or its analogue to a patient undergoing such treatment in an amount effective to reduce the side effects.

2. The method of claim 1, wherein the side effects are EGFR-TKI induced adverse gastrointestinal effects.

3. The method of claim 2, wherein the side effects are EGFR-TKI induced intestinal cell damage or growth inhibition.

4. The pharmaceutical composition of claim 1, wherein the EGFR-TKI is gefitinib (N-(3-Chloro-4-fluoro-phenyl)-7-methoxy-6-(3-morpholin-4-ylpropoxy)quinazolin-4-amine).

5. The method of claim 1, wherein the analogue is selected from the group consisting of

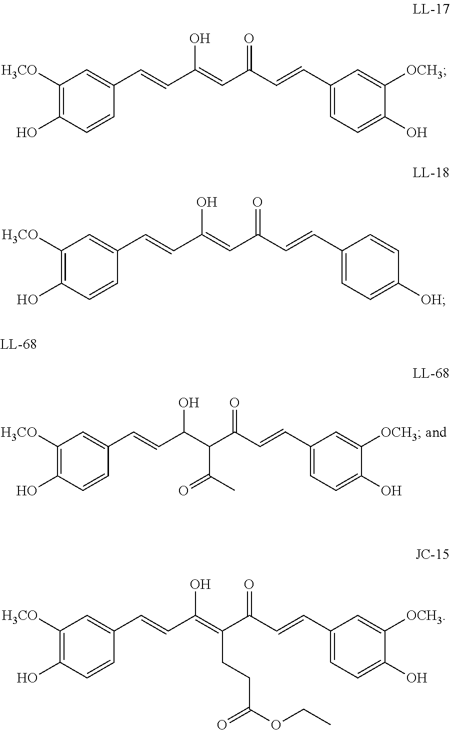

6. The method of claim 1, wherein the patient is afflicted with non-small-cell lung cancer (NSCLC).

7. The method of claim 1, wherein the curcumin or its analogue is administered concurrently with the EGFR-TKI.

8. A method for administering an epidermal growth factor receptor tyrosine kinase inhibitor (EGFR-TKI) to a patient in need of a cancer therapy using the EGFR-TKI, comprising administering to the patient a reduced dose of the EGFR-TKI in combination with curcumin or its analogue while efficacy of the EGFR-TKI with respect to the cancer therapy is substantially maintained as compared to that achieved with a standard dose of the EGFR-TKI without administration of the curcumin or its analogue.

9. The method of claim 8, wherein the reduced dose is about 50% or less of the standard therapeutic dose of the EGFR-TKI.

10. The method of claim 8, wherein the patient is afflicted with NSCLC.

11. The method of claim 8, wherein the patient is resistant to treatment with the EGFR-TKI alone.

12. The pharmaceutical composition of claim 8, wherein the EGFR-TKI is gefitinib (N-(3-Chloro-4-fluoro-phenyl)-7-methoxy-6-(3-morpholin-4-ylpropoxy)quinazolin-4-amine).

13. The method of claim 8, wherein the analogue is selected from the group consisting of:

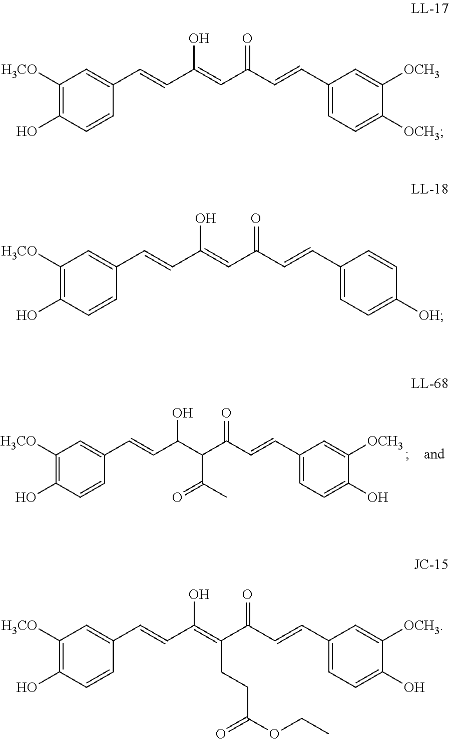

14. The method of claim 8, wherein the curcumin or its analogue is administered concurrently with the EGFR-TKI.

15. A method for treating a cancer patient with resistance to an EGFR-TKI, which comprises jointly administering to said patient an effective amount of the EGFR-TKI with curcumin or its analogue.


SEQUENCE LISTING (1 sequence)

SEQ ID NO: 1 - protein, 1210 aa, Homo sapiens [sequence omitted]